(19) United States
(12) Patent Application Publication
(10) Pub. No.: US 2020/0397607 A1
(43) Pub. Date: Dec. 24, 2020

(54) HAND ORTHOSIS AND SYSTEM HAVING A HAND ORTHOSIS

(71) Applicant: Ottobock SE & Co. KGaA, Duderstadt (DE)

(72) Inventors: Alexander HARDT, Aßlar (DE); Guido KAHLMEYER, Siemerode (DE); Klaus LIDOLT, Duderstadt (DE); Johan Fredrik FLOOD, Katrineholm (SE); Julia BIALOWONS, Göttingen (DE)

(73) Assignee: Ottobock SE & Co. KGaA, Duderstadt (DE)

(21) Appl. No.: 15/733,124

(22) PCT Filed: Nov. 16, 2018
(86) PCT No.: PCT/EP2018/081627

§ 371 Date: May 21, 2020

(30) Foreign Application Priority Data
Nov. 24, 2017 (DE) 10 2017 127 892.3

(51) Int. Cl. A61F 5/01; A63B 23/16; A63B 21/02

(52) U.S. Cl. CPC A61F 5/0118; A63B 21/028; A63B 23/16

(57) ABSTRACT

A hand orthosis having a cuff for fixing the hand orthosis to a wrist joint or a lower arm of a wearer of the hand orthosis, at least one support brace that extends radially or ulnarly next to a hand of the wearer in the direction of an extension of the lower arm, at least one carrier element arranged on the support brace, and a functional element detachable fixed to the carrier element.

The invention relates to a hand orthosis with a cuff for fixing the hand orthosis to a wrist or a lower arm of a wearer of the hand orthosis, at least one support brace that extends radially or ulnarly next to the hand of the wearer in the direction of an extension of the lower arm, and at least one carrier element that is arranged on the support brace. The invention also relates to a system with this type of hand orthosis.

Hand orthoses have been known within the scope of the prior art for many years. They feature a cuff with which the hand orthosis is arranged on the wrist or the lower arm of the wearer. The design of this cuff is different depending on the intended purpose of the hand orthosis. There are cuffs which only extend over the wrist and a small part of the lower arm so that the supporting effect remains small, for example if a thumb is to be supported. If the wrist is to be immobilized, a larger cuff is required which can also comprise rigid or less flexible elements, such as splints or rods.

With a number of hand orthoses, the fingers on the hand can be moved freely, even if it is not absolutely necessary. In many cases, the hand should be supported or, for instance, prevented from experiencing spasticities.

In the case of the hand orthosis according to the invention, a support brace extends in the direction of extension of the lower arm. This support brace is arranged next to the hand, i.e. radially or ulnarly, and can be arranged directly on the cuff, for example, or on a separate support element. This type of support element, which may also be designed to form a single piece with the support brace, is, for example, a rail element or a brace which is, for instance, at least partially, but preferably completely, guided around the wrist or the lower arm of the wearer of the hand orthosis.

A carrier element is situated on this support brace. The support brace extends in the direction of extension of the lower arm. However, this does not mean that the support brace and the lower arm extend parallel to one another in the mathematical sense. Rather, the support brace extends ulnarly or radially next to the hand.

A carrier element is arranged on the support brace, wherein said carrier element preferably extends at a right angle to the support brace. Of course, other angular settings are also possible. In this case, the carrier element itself preferably extends over the back of the hand, i.e. dorsally, or along the hand surface, i.e. volarly. Here, it extends from either the radial or ulnar side, where the support brace is situated, towards the respective opposite side.

Such hand orthoses can be used for a range of purposes. It is thus only possible to immobilize the wrist or to limit the wrist angle at which the wrist can be moved. Alternatively or additionally, spasticities can be prevented or dexterity trained following an operation, for example. The prior art provides different types of hand orthoses for all of these different requirements.

The invention therefore aims to further develop the type of hand orthoses initially referred to in such a way that warehousing requirements can be reduced and the flexibility regarding the applicability of the hand orthosis can be increased. The invention solves the problem by way of a hand orthosis according to the generic term in claim 1, characterized in that a functional element is fixed on the carrier element such that it can be detached.

This means that a base body of the hand orthosis with a cuff, support brace and carrier element can be used for a variety of different functions and that the elements which actually serve to meet the requirements can be fixed to the carrier element in the form of the functional element. Consequently, it is no longer necessary to provide different hand orthoses for different functions, as is the case with the prior art; rather, only different functional elements need be provided, while the remaining part of the hand orthosis can be used, regardless of the intended use. This reduces warehousing requirements, which allows costs to be saved. In addition, different functional elements can be used in succession, for example during the healing process following an operation, without the patient having to acquire different hand orthoses.

Preferably, the functional element is arranged on the carrier element in a positive-locking manner, in particular by clipping it onto or plugging it onto the carrier element or by fixing it with at least one velcro element. This renders a change and adjustment especially easy.

In a preferred configuration, the carrier element features, for instance, a positive-locking element, while the functional element comprises the corresponding counter-element. The two positive-locking elements can interact with one another such that a positive-locking connection is established, for example by a part of the positive-locking element of the carrier element locking or snapping into a specially provided recess in the counter positive-locking element on the functional element. The reverse scenario is of course also possible. An activation element can be used to disengage the thus established snap or clip connection and the functional element removed from the carrier element.

Alternatively or additionally, the functional element has a recess into which the carrier element can be inserted. This allows the functional element to be plugged onto the carrier element. Here, the functional element may also comprise a locking device, such as a screw or a clamping device, with which the plugged-on functional element can be fixed to the carrier element so as to prevent slipping or displacement. The individual position in which the functional element is arranged on the carrier element can therefore be easily and infinitely adjusted. By marking the carrier element and/or the functional element, for example, reproducibility as well as a reduction in operating errors can be achieved.

In this case, the carrier element preferably has a non-circular cross-section. In addition, the recess in the functional element preferably has the same cross-section. As a result, the functional element can only be plugged onto the carrier element in very specific orientations, preferably in only one orientation. This prevents operating errors from occurring. Of course, it is also possible to provide both the carrier element and the recess of the functional element with a circular cross-section. In this case, the functional element can be rotated about a longitudinal axis of the carrier element, i.e. the center of the respective cross-section. The orientation of the functional element relative to the hand can thus be adjusted more effectively.

Preferably, the functional element refers to a volar support element or a dorsal support element. A volar support element is arranged on the palm of the hand, i.e. the inner surface of the hand. It is used, for example, to prevent spasticities, i.e. cramping of the hand to form a fist. Alternatively, the functional element may also be a dorsal support element. A dorsal support element is arranged on the back of the hand and can exert a tensile force on the fingers, for example, thereby counteracting the cramping of the hand to form a fist. Of course, elastic support elements can also be used in this case: here, said elements do not fully prevent a movement of the fingers, but rather make such a movement more difficult against the spring force.

Preferably, the support brace can be fixed on a support element in a first position or a first orientation and in at least a second position or at least a second orientation. Alternatively or additionally, the carrier element can be fixed on a support element in a first position or a first orientation and in at least a second position or at least a second orientation. Here, the support brace and/or the carrier element are preferably shaped in such a way that the carrier element is positioned dorsally to the hand when the support brace and/or the carrier element is in the first position or orientation. This is an advantage if a force or an effect is to be exerted by the functional element on the back of the hand or on the fingers from the same direction.

Conversely, the carrier element is preferably arranged to be volar, i.e. in the palm region, when the support brace and/or the carrier element is in a second position or a second orientation. This allows functional elements to be arranged on the inner surface of the hand in order to thereby produce the desired effect.

The functional element preferably features at least one corrective device. Preferably, corrective devices are provided for several fingers, preferably for each individual finger. Alternatively or additionally, the functional element preferably comprises a hand shell or a hand brace. This hand shell or hand brace can be designed as a volar or dorsal support element and support the hand from the specified direction. Alternatively or additionally, the functional element features a training device by way of which, for example, the motor skills of the hand can be restored and trained, especially following a surgical procedure. A training device preferably refers to a training device that can be elastically deformed, such as a ball. Of course, other shaped training devices are possible.

The invention also solves the problem by way of a system with a hand orthosis as described here and at least a second functional element that can be fixed to the carrier element such that it can be detached. In this case, the second functional element is a different functional element to the first functional element, which forms part of the hand orthosis.

A hand orthosis of the type described here may be part of an arm or even a shoulder orthosis. Such an orthosis comprises, for example, a shoulder orthosis element, which has an upper arm section. A fixing element, such as a fixing belt or strap, may be fixed to this upper arm section; the hand orthosis can be arranged on said fixing element. Of course, two or more fixing elements, such as fixing straps, may be provided. The hand orthosis can preferably be fixed to the shoulder section such that it can be detached, for example using press studs or clasps. Here, it is also an advantage if no distinction between right and left shoulder elements has to be made; rather, the hand orthosis can be used for both sides.

Such a shoulder orthosis is used for patients with shoulder complaints or shoulder dysfunctions, for instance, following a stroke or injuries to the central or peripheral nervous system. The hand orthosis enables an even better treatment of the affected patients.

The hand orthosis can also be used without a functional element. The carrier element is adjustably arranged on the support brace in the manner described. The lower hand can be positioned via the carrier element. It can be used in particular in combination with a shoulder orthosis; however, it can also without being arranged on other components. It can be used to position the lower hand of the wearer and particularly to exert a rotation (“twisting effect”).

In the following, examples of embodiments of the present invention will be explained in more detail by way of the attached figures: They show:

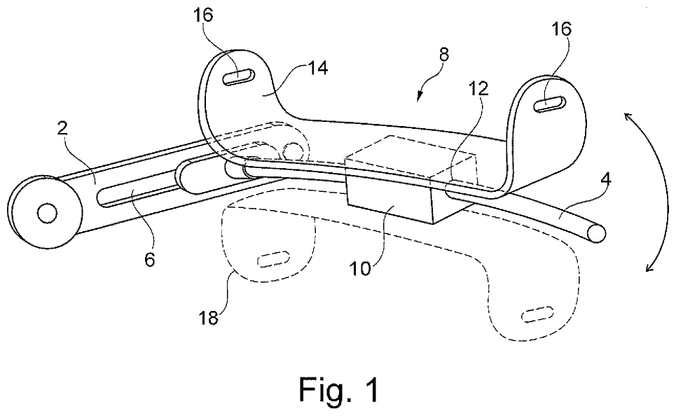

FIGS. 1 to 4—schematic representations of a part of a hand orthosis according to examples of embodiments of the present invention,

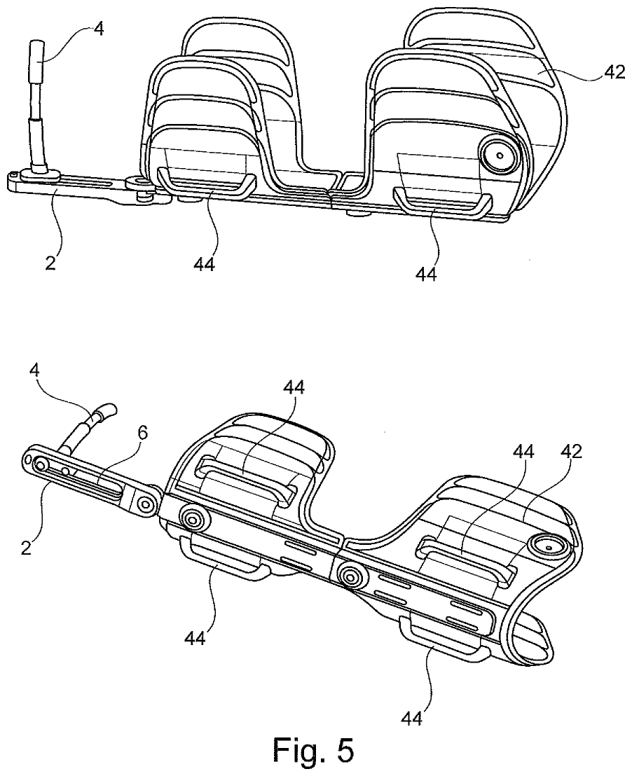

FIG. 5—schematic representations of a part of a hand orthosis and

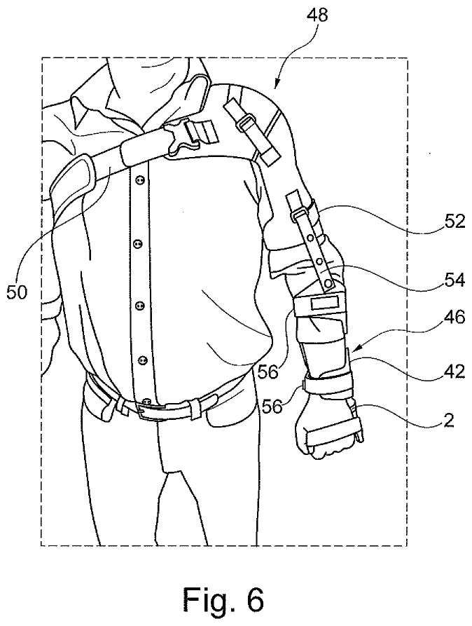

FIGS. 6 to 8—representations of an orthosis in the applied state.

FIG. 1 depicts a part of a hand orthosis according to a first example of an embodiment of the present invention.

There is a carrier element 4 with a support brace 2, which is arranged on a sleeve not depicted here; in the example of an embodiment shown, the carrier element has a circular cross-section. The carrier element 4 is arranged in a specially provided elongated hole 6 on the support brace 2 such that it can be displaced. This allows for an adjustment of the position of the carrier element 4 relative to the hand of the wearer of the hand orthosis.

A functional element 8 is plugged onto the carrier element 4, said functional element featuring a box-shaped fixing device 10 that comprises a recess 12 which extends through the fixing device 10 and also has a circular cross-section. The fixing device 10 can be fixed to the carrier element 4 via a locking element, not depicted here, such as a screw or a clamping device.

In the example of an embodiment shown, the functional element 8 has a hand brace 14, in which, for example, a part of the hand of the wearer can be laid. At its ends, which curve upwards, are two openings 16; a belt can be threaded through each of these openings to fix the hand to the hand brace 14. Due to the circular cross-section of both the carrier element 4 and the recess 12, the hand brace 14 can also be swivelled out of the depicted volar position, in which the hand in FIG. 1 is inserted into the hand brace 14 from above, into a dorsal position. This is shown via the dashed line 18. In this position, the functional element 8 can also be fixed to the carrier element 4 by means of the fixing device 10. In this case, the hand brace 14 is placed on the back of the hand of the wearer from above.

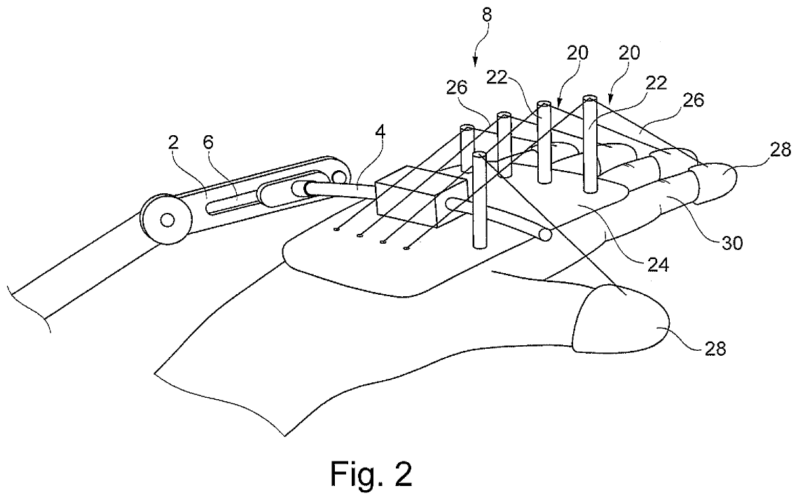

FIG. 2 shows an alternative configuration. Here, the carrier element 4 is also designed with a circular cross-section and can be displaced in the elongated hole 6 of the support brace 2. In this case, the functional element 8 is designed with corrective devices 20. Such a corrective device features a pin 22, which is arranged on a base plate 24; each pin acts as a carrier for one corrective wire 26. A corrective wire 26 does not necessarily refer to a wire made of metal, even though this represents one possible configuration. The corrective wire 26 may also be designed to be a thread or a cable or another tension element. The corrective wire 26 is preferably designed to be elastic. One end of the corrective wire is fixed to the base plate 24, while in the example of an embodiment shown a finger cap 28 is situated at the respective other end of the corrective wire 26, wherein said finger cap can be pulled over one of the fingers 30. In this case, a tensile force is exerted via the corrective wire 36 on the finger tip of the finger 30 on which the finger cap 28 is situated, thereby preventing or at least restricting a movement of the finger 30.

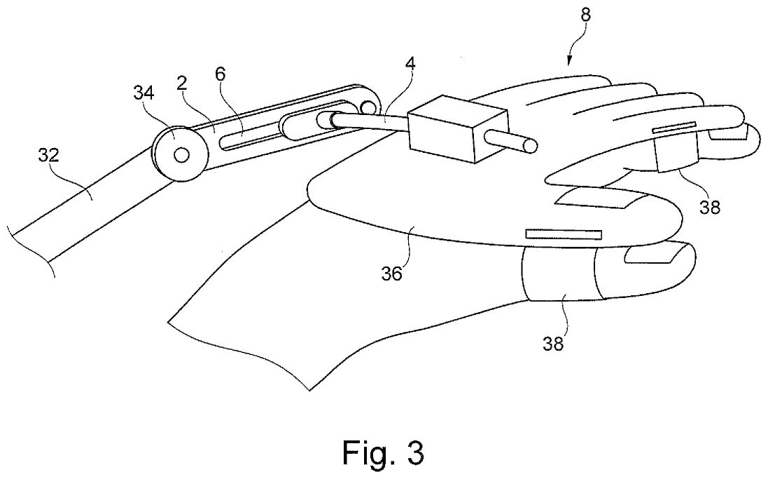

FIG. 3 shows another configuration. Here, the carrier element 4 is also designed with a circular cross-section and is positioned in the elongated hole 6 on the support brace 2 such that it can be displaced. The support brace 2 is arranged on a support element 32 and can be brought into different orientations via a swivel joint 34. In the example of an embodiment shown, the functional element 8 is designed as a support plate 36. It is arranged dorsally on the hand and comprises finger sleeves, through which the individual fingers can be arranged on the support plate 36.

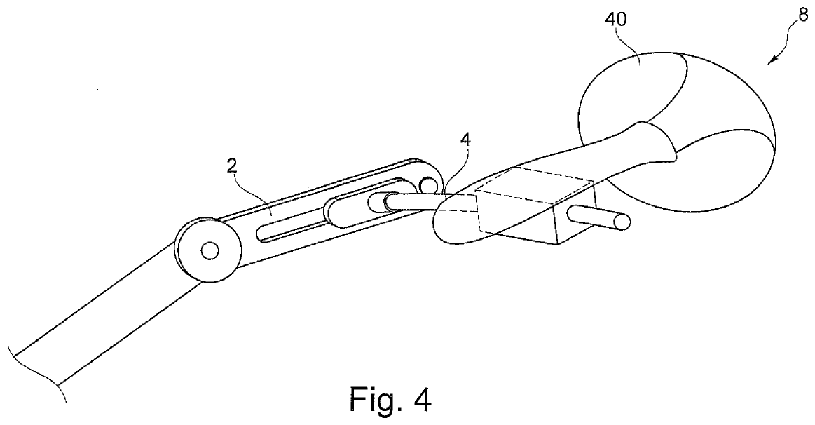

FIG. 4 depicts a further example of an embodiment. In the example of an embodiment shown, the functional element 8 is a training ball 40, which is preferably designed to be elastic and serves to train the hand. The carrier element 4 and the support brace 2 are designed in the same way as in the other examples of embodiments.

Both representations in FIG. 5 depict a three-dimensional schematic view of a part of the hand orthosis. The carrier element 4 is arranged at the end of the support brace 2 in which the elongated hole 6 is situated. The carrier element is designed to be curved and can be completely removed from the elongated hole 6 and inserted in the reverse orientation. This renders it especially easy to distinguish between a hand orthosis for the right side and one for the left. The hand orthosis features a shell element 42, on which loops 44 are arranged, through which the belts—not depicted here—are guided in order to attach the hand orthosis to the lower arm of the wearer. Together with the shell element, these form the cuff of the hand orthosis. In the example of an embodiment shown, the shell element 42 comprises two parts which can be displaced relative to one another. By displacing the two parts relative ro one another, the length can be adjusted.

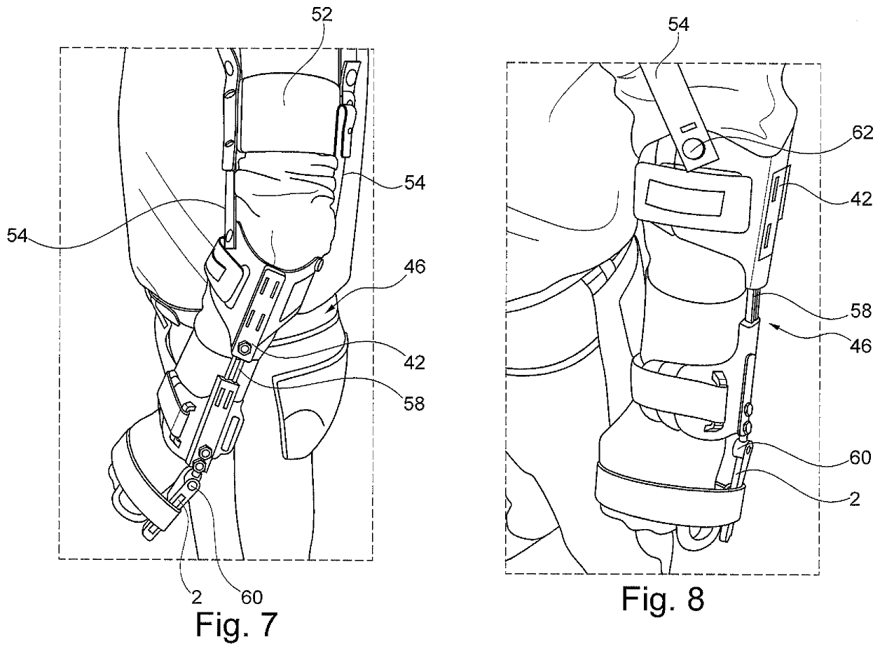

FIGS. 6 to 8 show an orthosis in the applied state. It features a hand orthosis 46 according to an example of an embodiment of the present invention. FIG. 6 shows that the orthosis comprises a shoulder section 48, which is arranged via a strap 50 on the torso of the wearer. The shoulder section features an upper arm cuff 52, at the lower end of which two connecting straps 54 are arranged on which the hand orthosis 46 is situated. Said hand orthosis comprises several straps 56, which are arranged on the shell element 42 and, together with this shell element, form the cuff. In the example of an embodiment shown, the support brace 2 extends ulnarly next to the hand in the direction of an extension of the lower arm of the wearer; the carrier element 4, not depicted in FIG. 6, is situated on said support brace. FIGS. 7 and 8 depict enlarged detailed representations from different perspectives. In FIG. 7, the two connecting straps 54 can be clearly recognized, by way of which the hand orthosis 46 is arranged on the upper arm cuff 52. The shell element 42 comprises a length adjustment unit 58 so as to enable the optimal length for the length of the lower arm of the wearer to be set. In the example of the embodiment shown, the support brace 2, which extends as an extension of the lower arm, is arranged on the shell element 42 via a hinge joint 60 in order to enable an optimal angular adjustment at this point as well. In FIG. 8, it is also clear that the shell element 42 is connected to the connecting straps 54 via a press stud 62. As a result, it is especially easy to detach the hand orthosis 46 from the upper arm cuff 52 of the shoulder section 48.

### REFERENCE LIST

2 support brace
4 carrier element
6 elongated hole
8 functional element
10 fixing device
12 recess
14 hand brace
16 opening
18 dashed line
20 corrective device
22 pin
24 base plate
26 corrective wire
28 finger cap
30 finger
32 support element
34 swivel joint
36 support plate
38 finger sleeve
40 training ball
42 shell element
44 loop
46 hand orthosis
48 shoulder section
50 strap
52 upper arm cuff
54 connecting strap
56 strap
58 length adjustment unit
60 hinge joint
62 press stud

1. A hand orthosis comprising:
a cuff for fixing the hand orthosis to a wrist joint or a lower arm of a wearer of the hand orthosis;
at least one support brace, which extends radially or ulnarly next to a hand of the wearer in a direction of an extension of the lower arm;
at least one carrier element, which is arranged on the at least one support brace;
a functional element is fixed to the carrier element such that the functional element can be detached.

2. The hand orthosis according to claim 1, wherein the functional element is arranged on the carrier element in a positive-locking manner.

3. The hand orthosis according to claim 1, wherein the functional element is a volar support element or a dorsal support element.

4. The hand orthosis according to claim 1, wherein the at least one support brace can be fixed on a support element in a first position or a first orientation and in at least a second position or at least a second orientation.

5. The hand orthosis according to claim 1, wherein the functional element comprises at least one corrective device.

6. The hand orthosis according to claim 1, wherein the functional element comprises a hand shell or a hand brace.

7. The hand orthosis according to claim 1, wherein the functional element comprises a training device.

8. A system with a hand orthosis according claim 1, and at least a second functional element, which can be fixed to the carrier element such that the at least the second functional element can be detached.

9. The hand orthosis according to claim 2, wherein the positive-locking manner includes clipping or plugging the functional element onto the carrier element or connecting the functional element to the carrier element with at least one Velcro® element.

10. The hand orthosis according to claim 7, wherein the training device is elastically deformable.

11. A hand orthosis comprising:
a cuff configured to fix the hand orthosis to a wrist joint or a lower arm of a wearer of the hand orthosis;
at least one support brace configured to extend radially or ulnarly next to a hand of the wearer in a direction along a length of the lower arm;
at least one carrier element arranged on the at least one support brace;
a functional element detachable mounted to the carrier element.

12. The hand orthosis according to claim 11, wherein the functional element is arranged on the carrier element in a positive-locking manner.

13. The hand orthosis according to claim 12, wherein the positive-locking manner includes clipping or plugging the functional element onto the carrier element or connecting the functional element to the carrier element with at least one Velcro® element.

14. The hand orthosis according to claim 11, wherein the functional element is a volar support element or a dorsal support element.

15. The hand orthosis according to claim 11, wherein the at least one support brace is mounted on a support element in a first position or a first orientation or in at least a second position or at least a second orientation.

16. The hand orthosis according to claim 11, wherein the functional element comprises at least one corrective device.

17. The hand orthosis according to claim 11, wherein the functional element comprises a hand shell or a hand brace.

18. The hand orthosis according to claim 11, wherein the functional element comprises a training device.

19. The hand orthosis according to claim 18, wherein the training device is an elastically deformable ball.

20. A system comprising:
a hand orthosis according to claim 11;
at least a second functional element detachably mounted to the carrier element.
